(19) United States
(12) Patent Application Publication
(10) Pub. No.: US 2022/0336111 A1
(43) Pub. Date: Oct. 20, 2022

(54) SYSTEM AND METHOD FOR MEDICAL LITERATURE MONITORING OF ADVERSE DRUG REACTIONS

(71) Applicant: The Provost, Fellows, Foundation Scholars and the other members of Board, of the College of the Holy, Dublin 2 (IE)

(72) Inventors: Bruno Ohana, Dublin 2 (IE); Lucy Hederman, Dublin 2 (IE); Nicole Baker, Dublin 2 (IE)

(73) Assignee: The Provost, Fellows, Foundation Scholars and the other members of Board, of the College of the Holy, Dublin 2 (IE)

(21) Appl. No.: 17/725,486

(22) Filed: Apr. 20, 2022

Related U.S. Application Data
(60) Provisional application No. 63/177,352, filed on Apr. 20, 2021.

(51) Int. Cl. G16H 70/40; G06F 16/2455

(52) U.S. Cl. CPC G16H 70/40; G06F 16/2455

(57) ABSTRACT

A system and method for medical literature monitoring of adverse drug relations, enabled by screening literature references by applying one or more machine learning models trained using a data labelling protocol and a plurality of data rules prescribed by a plurality of subject matter experts. The data labelling protocol comprises a set of inferences derived from screening and labelling a plurality of medical literature with suspected references to adverse drug reactions by subject matter experts. Suspected references to adverse drug reactions includes direct references to adverse drug reactions and indirect references to adverse drug reactions. The plurality of data rules is derived from observations of subject matter experts during data labelling. The predictions outputted by each of the machine learning models are validated with the data rules, and a final list of literature with suspected references to adverse drug reactions is generated.

This patent application claims the benefit of U.S. Provisional Patent Application Ser. No. 63/177,352, filed 20 Apr. 2021, the specification of which is hereby incorporated herein by reference.

### BACKGROUND

### Field of the Invention

The present disclosure relates to a system and method for medical literature monitoring of adverse drug reactions.

### DESCRIPTION OF THE RELATED ART

Medical literature monitoring (MLM) of adverse drug reactions is an important aspect of the pharmacovigilance process. MLM is also a regulatory requirement for marketed medicinal products.

The main purpose of the MLM process is to identify and report adverse events from published literature. The output of the MLM process is a subset of the input articles containing one or more confirmed adverse events relating to the product of interest. Typically, scientific databases provide only the abstract and title of articles, followed by metadata such as author, journal name, etc. The full text of an article may only be available upon purchase and it would be costly to purchase full text versions of all articles obtained from a search. Hence Medical Literature Monitoring of adverse drug reactions is usually performed as a two-stage process, wherein the abstracts of the input articles are first screened based on relevant references to adverse events, and thereafter a detailed evaluation of the full text of the candidate articles obtained from the first screen is performed.

In the MLM process, a positive identification of an article carrying an adverse event is obtained when the article matches all four required criteria for an Individual Case Safety Report (ICSR). These can be: (1) the article contain an identified source (i.e. the authors), (2) one or more identifiable patients, (3) the article discusses the product of interest and (4) the article describes an adverse drug reaction with a causal link to the product of interest.

MLM is an extremely time-consuming task since it requires reviewing and filtering of voluminous amounts of literature which may or may not contain references to adverse drug reactions. This also requires specialist knowledge since only a small fraction of the reviewed literature become valid individual case safety reports (ICSRs).

While removing irrelevant literature is desired for efficiency purposes, it is far more important to maintain very low false negative rates, that is incorrectly flagging an adverse event article as irrelevant. Non-detection of a valid ICSR (a false negative) carries a high cost in auditing and rework, while detecting a non-event as adverse (a false positive) incurs only an incremental screening cost. Therefore, automated methods used for screening literature for adverse events must show high recall when identifying adverse articles, even at the expense of precision.

Prior art methods and systems screen literature for adverse drug reactions using manual or computer assisted methods that require human involvement to review all inbound articles. Such methods are often time consuming and suffer from lack of accuracy and efficiency.

There is therefore an unresolved and unfulfilled need in the art for a system and method for medical literature monitoring of adverse drug reactions, which automates the step of screening literature with references to relevant adverse drug reactions using inputs from subject matter experts, and this forms the primary objective of at least one embodiment of the invention.

### BRIEF SUMMARY

Embodiments of the invention, as set out in the appended claims, relates to a system and method for medical literature monitoring of adverse drug relations, enabled by screening literature references by applying one or more machine learning models trained using a data labelling protocol and a plurality of data rules prescribed by a plurality of subject matter experts.

In at least one embodiment of the invention a method for medical literature monitoring of adverse drug reactions is presented. The method comprises the steps of searching one or more databases consisting medical literature with references to adverse drug reactions to one or more medications and generating a plurality of search results. The plurality of search results are screened and one or more literature references with suspected relevant references to adverse drug reactions are shortlisted from the search results for further review by applying one or more trained machine learning models. The machine learning models are trained using a data labelling protocol and a plurality of data rules prescribed by a plurality of subject matter experts, and the suspected references to adverse drug reactions includes direct references to adverse drug reactions and indirect references to adverse drug reactions. The predictions outputted by the machine learning models are validated with the plurality of data rules, and a final list of literature with suspected references to adverse drug reactions are generated based on the validated predictions.

In one embodiment of the invention, a system for medical literature monitoring of adverse drug reactions is presented. The system comprises a computing device and a memory means operably coupled to the computing device. The memory means has a plurality of instructions stored thereon which configures the computing device to train one or machine learning models using a data labelling protocol and a plurality of data rules prescribed by a plurality of subject matter experts; generate a plurality of search results by searching one or more databases consisting medical literature with reference to adverse drug reactions to one or more medications; apply the machine learning models to screen the search results, the screened literature references consisting literature with suspected references to adverse drug reactions; validate predictions outputted by the one or more machine learning models with the plurality of data rules; and generate a list of literature with suspected references to adverse drug reactions based on the validated predictions.

In an embodiment of the invention, the predictions of the machine learning models which are in conflict with the data rules are discarded.

In an embodiment of the invention, the data labelling protocol comprises a set of inferences (or rules) derived from screening and labelling a plurality of medical literature with suspected references to adverse drug reactions by the subject matter experts.

In an embodiment of the invention, the machine learning models are continuously reinforced and improved using the validated predictions and the generated list of literature references.

In an embodiment of the invention, text encoding errors and additional meta tags such as HTML tags are removed from the search results.

In an embodiment of the invention, text in the search results is converted into features capable of being inputted to the one or more machine learning models.

At least one embodiment of the invention hence provides a robust and cost-effective solution to problems identified in the art, by applying machine learning models as a first-pass filter to remove irrelevant articles thereby addressing high screening volumes in MLM.

### BRIEF DESCRIPTION OF DRAWINGS

At least one embodiment of the invention will be more clearly understood from the following description of an embodiment thereof, given by way of example only, with reference to the accompanying drawings, in which:

FIG. 1 is a flow diagram illustrating a method as per an embodiment of the invention.

FIG. 2 is a Venn diagram illustrating contents of literature with suspected references to adverse events.

FIG. 3 is a graphical representation illustrating savings due to filtering irrelevant articles for a predefined target value of recall, as per an embodiment of the invention.

FIG. 4 illustrates a model architecture description showing the different modules according an embodiment of the invention.

### DETAILED DESCRIPTION OF THE INVENTION

At least one embodiment of the invention relates to a system and method for medical literature monitoring of adverse drug reactions, and more particularly to a system and method for medical literature monitoring of adverse drug relations, enabled by screening literature references by applying one or more machine learning models trained using a data labelling protocol and a plurality of data rules, prescribed by a plurality of subject matter experts.

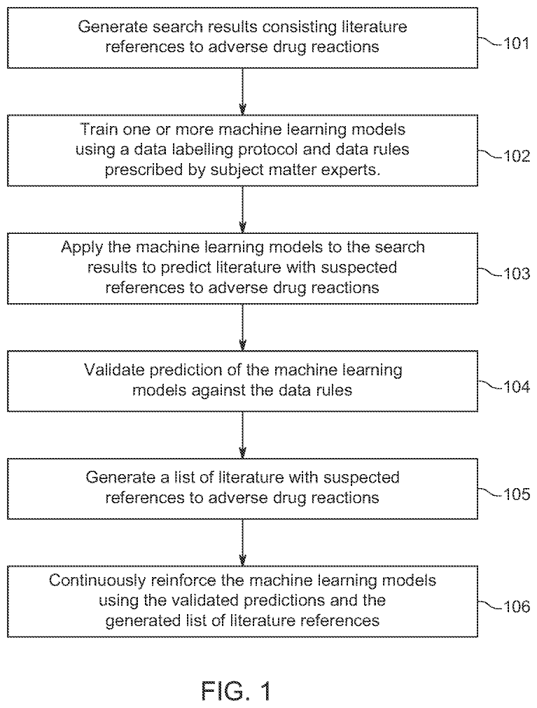

Referring to FIG. 1, the method as per at least one embodiment of the invention comprises the first step of performing a search in one or more databases with medical literature with references to adverse drug reactions to one or more medications 101. The databases are searched using search queries which consists of for example, names of medicines of interest, related synonyms, and brand names of interest. A plurality of search results is generated, and the search results are de-duplicated in case the same result has been retrieved previously or the same entry is outputted from multiple databases previously searched. The text in the search results is normalized by removing common issues such as encoding errors, metatags, and other unwanted content. Further, the text in the search results is converted into features capable of being inputted to one or more machine learning models.

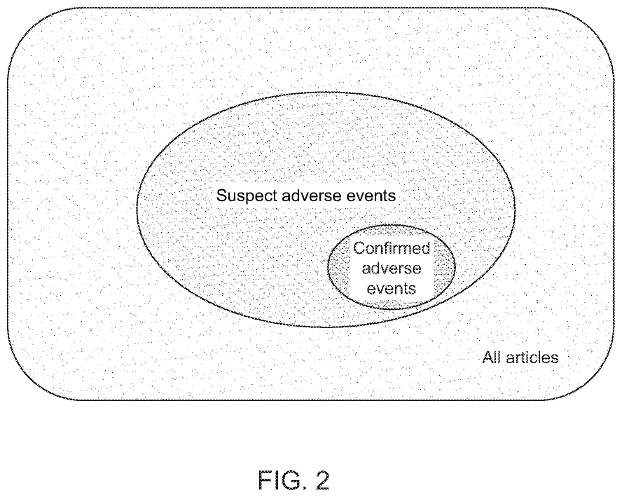

The resulting search results are screened for one or more literature references with suspected references to adverse drug reactions by applying one or more trained machine learning models 103. Suspected references to adverse drug reactions include direct references to adverse drug reactions and indirect references to adverse drug reactions as illustrated in FIG. 2. For example, a direct reference to an adverse drug reaction in the abstract of an article may read “We describe the case of a 43 year old male patient suffering from headaches following treatment with benzodiazepine”. An indirect reference to an adverse event only describes adverse event in the full text of the article and not in the abstract, for example an indirect reference may read—“We report on the results of a series of cases being treated for rheumatoid arthritis with methotrexate, where only mild adverse reactions were observed.”.

The machine learning models are trained using a data labelling protocol and a plurality of data rules prescribed by a plurality of subject matter experts 102. The data labelling protocol comprises a set of inferences derived from screening and labelling a plurality of medical literature with suspected references to adverse drug reactions by subject matter experts. The prior pharmacovigilance know-how of subject matter experts is leveraged to generate labelled data in the form of articles containing suspected adverse events which serves as raw input for training the machine learning models. When labelling of literature is performed by subject matter experts to generate training data, it is not possible to always specify what treatments are implicated in the suspect event. This approach to data labelling can be considered as a trade-off between precision, i.e., only detecting direct adverse events, and recall. High recall is emphasized to minimize the risk of missing references to potential adverse events.

The plurality of data rules is derived from observations of subject matter experts during data labelling. Information extracted from the search results such as references to patients, medicines, or therapies, is also used for framing the data rules. The data rules complement and act as a safeguard preventing the machine learning models from making erroneous predictions in case certain patterns cannot be easily learnt from the data labelling protocol, thus increasing recall.

The predictions outputted by the machine learning models are validated against the data rules 104. The predictions of the machine learning models which conflict with the data rules, are discarded. The data rules override machine learning behaviour with the aim of building higher quality or safer predictions. They are compiled using logic predicates that treat previously generated artifacts as facts. Rules combine the output of the machine learning model and the information extracted from raw text. For example, there may be a rule which reads: If [text contains patient mention] and [Prediction score for suspect adverse machine learning model>0.4] THEN SET document prediction=“suspect adverse”.

Training the machine learning models with the data labelling protocols and validating the predictions of the machine learning models using the plurality of data rules enables to replicate expertise of subject matter experts to perform MLM and also leverages the domain knowledge of subject matter experts for more robust predictions.

Based on the validated predictions of the machine learning models, a list of literature with suspected references to adverse drug reactions is generated 105. The machine learning models are continuously reinforced using the validated predictions and the generated list of literature references 106.

FIG. 2 is a Venn diagram illustrating contents of literature with suspected references to adverse events.

In at least one embodiment of the invention, a system for medical literature monitoring of adverse drug reactions is presented. The system comprises a computing device and a memory means operably coupled to the computing device. The memory means may be any internal or external device or web-based data storage mechanism adapted to store data. The computing device may be a personal computer, a portable device such as a tablet computer, a laptop, a smart phone, connected medical device or any operating system based connected portable device.

The memory means has a plurality of instructions stored thereon which configures the computing device to train one or machine learning models using a data labelling protocol and a plurality of data rules prescribed by a plurality of subject matter experts. The computing device is configured to generate a plurality of search results by searching one or more databases consisting medical literature with reference to adverse drug reactions to one or more medications. The machine learning models are then applied to screen the search results wherein the screened literature references consist of literature with suspected references to adverse drug reactions. Each machine learning model outputs an independent prediction and each prediction is validated with the plurality of data rules. A list of literature references with suspected references to adverse drug reactions is then generated based on the validated predictions.

The computing device is further configured to remove encoding errors and metatags from the search results; convert text in the search results into features capable of being inputted to the one or more machine learning models; and extract information from the search results for framing the plurality of data rules.

In at least one embodiment of the invention, literature screening was performed for a dataset of article metadata, and references to suspected adverse events in the dataset was predicted. The dataset was split by months, March to June. The prediction threshold for a desired recall was calibrated based on the previous month's data. Table 1 illustrates the results obtained when the desired recall was predefined as 95%.

As shown in Table 1, for a desired recall of 95%, savings in excess of 40% was obtained due to filtering of irrelevant articles.

TABLE 1

| Calibration | Target | Recall- | Recall- | % Articles |
|---|---|---|---|---|
| Month | Month | Calibration | Target | filtered |
| February | March | 95.5 | 94.7 | 47% |
| March | April | 95.0 | 94.9 | 41% |
| April | May | 95.3 | 97.2 | 40% |
| May | June | 95.1 | 96.1 | 49% |

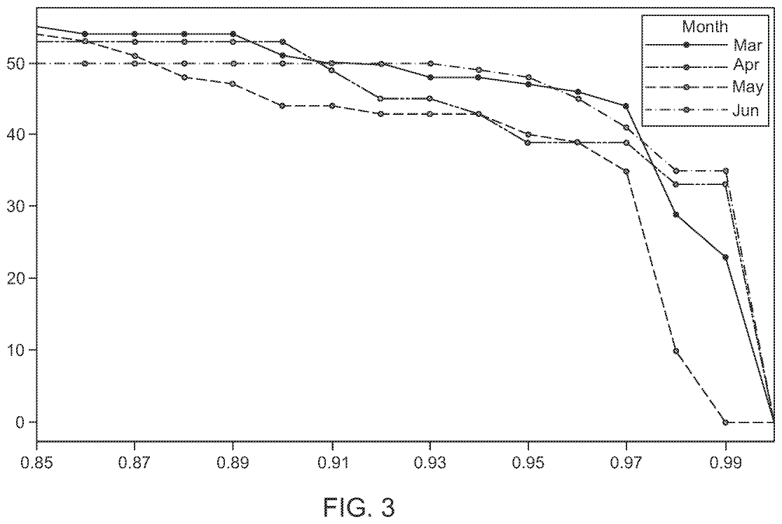

FIG. 3 illustrates the savings due to filtering irrelevant articles for higher values of recall for datasets corresponding to each month.

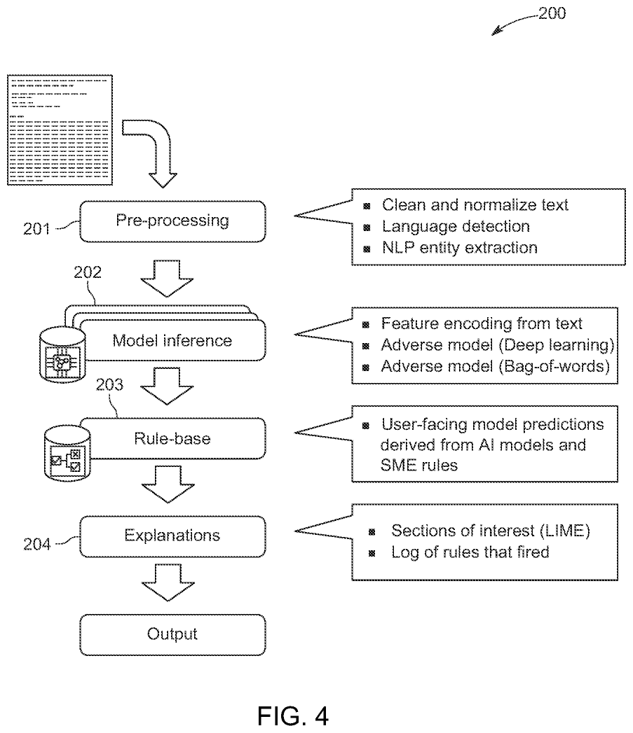

FIG. 4 illustrates a model description showing the different modules according an embodiment of the invention, indicated generally by the reference numeral 200. The inference pipeline supporting the adverse model comprises a pre-processing stage 201 where input text is cleaned and tokenized. This stage also performs language detection and a rule-based entity extraction of patient mentions which is used in later stages. A model inference stage 202 encodes the normalized text into features and runs the prediction step of the machine learning models, producing raw model predictions. Next, a post-processing rules-based stage 203 produces the final predictions and an explanation model stage 204 computes additional metadata that can be used for helping users in understanding model predictions.

The model inference stage 202 can use neural model which can employ a multi-layer neural network architecture organized as follows.

An initial embedding layer converts tokens into vector representations using a combination of pre-trained word embeddings built with an inputted biomedical text corpus [REF] and additional trainable embedding layers derived from part-of-speech tags and dependency parsing tags. The embeddings are combined and processed by a series of convolutional layers followed by a LSTM recurrent layer and an attention layer. Regularization is applied across the network architecture by using drop out during training and the use of batch normalization layers.

The neural model can be supplemented by a bag-of-words model using 1-gram and 2-grams as features and trained with a random forest estimator. During the rule-based inference stage 203, the neural and bag-of-word model predictions are combined and subject to override rules authored in conjunction with pharmacovigilance subject matter experts.

In drug safety, model mistakes have an asymmetric risk profile: articles falsely identified as a safety event (false positive) incurs incremental screening effort, while articles falsely identified as not a safety event (false negative) has a negative impact on what safety information is detected. Therefore false negatives are riskier and it is of paramount importance that this metric is minimized to ensure more accurate results.

To ensure the rate of false negatives remains statistically within bounds, an adverse event model is parametrized for a desired target recall level. With desired recall fixed at a sufficiently high level, a metric that reflects the additional effort caused by false positives should be minimized. At least one embodiment of the invention can use the false positive rate, defined as the ratio of false positives (FP) to the number of ground truth negative examples (N) given by:

$$\frac{FP}{N} = \frac{FP}{FP + TN}$$

Where FP is the number of false positives and TN is the number of true negatives. Thus, the performance target is the minimization of false positive rate at a desired target recall, set at 99%.

Test set results for experimental data, tuned for a 99% desired recall are shown below. All metrics are with respect to suspect adverse found class.

| Metric (adverse class) | Value |
|---|---|
| Recall | 98.8% |
| False Positive Rate | 45% |
| Precision | 57% |
| f1 score | 0.72 |

Although the at least one embodiment of the invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the invention as defined.

Further, a person ordinarily skilled in the art will appreciate that the various illustrative method steps described in connection with the embodiments disclosed herein may be implemented using electronic hardware, or a combination of hardware and software. To clearly illustrate this interchangeability of hardware and a combination of hardware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the invention.

In the specification, the terms “comprise, comprises, comprised and comprising” or any variation thereof and the terms “include, includes, included and including” or any variation thereof are considered to be totally interchangeable, and they should all be afforded the widest possible interpretation and vice versa.

1. A method for medical literature monitoring of adverse drug reactions, the method comprising the steps of:
searching one or more databases consisting medical literature with references to adverse drug reactions to one or more medications and generating a plurality of search results;
screening one or more literature references from the search results generated in step (a) by applying one or more trained machine learning models, the screened literature references consisting literature with suspected references to adverse drug reactions, wherein the one or more machine learning models are trained using a data labelling protocol and a plurality of data rules prescribed by a plurality of subject matter experts, and wherein the suspected references to adverse drug reactions includes direct references to adverse drug reactions and indirect references to adverse drug reactions;
validating predictions outputted by the one or more machine learning models, with the plurality of data rules; and
generating a list of literature with suspected references to adverse drug reactions based on the validation in step (c).

2. The method as claimed in claim 1, further comprising the step of discarding predictions which are in conflict with the plurality of data rules.

3. The method as claimed in claim 1, further comprising the step of continuously reinforcing the one or more machine learning models using the validated predictions and the generated list of literature references.

4. The method as claimed in claim 1 wherein the data labelling protocol comprises a set of inferences derived from screening and labelling a plurality of medical literature with suspected references to adverse drug reactions by the subject matter experts.

5. The method as claimed in claim 1 further comprising the steps of:
removing encoding errors and metatags from the search results; and
converting text in the search results into features capable of being inputted to the one or more machine learning models.

6. The method as claimed in claim 1, further comprising the step of extracting information from the search results for framing the plurality of data rules.

7. A system for medical literature monitoring of adverse drug reactions, the system comprising a computing device and a memory means operatively coupled to the computing device, the memory means having a plurality of instructions stored thereon which configures the computing device to:
train one or machine learning models using a data labelling protocol and a plurality of data rules, prescribed by a plurality of subject matter experts;
generate a plurality of search results by searching one or more databases consisting medical literature with reference to adverse drug reactions to one or more medications;
apply the machine learning models to screen the search results, the screened literature references consisting literature with suspected references to adverse drug reactions;
validate predictions outputted by the one or more machine learning models with the plurality of data rules; and
generate a list of literature with suspected references to adverse drug reactions based on the validated predictions.

8. The system as claimed in claim 7, wherein the suspected references to adverse drug reactions includes direct references to adverse drug reactions and indirect references to adverse drug reactions.

9. The system as claimed in claim 7, wherein the computing device is configured to discard predictions which are in conflict with the plurality of data rules.

10. The system as claimed in claim 7, wherein the computing device is further configured to continuously reinforce the one or more machine learning models using the validated predictions and the generated list of literature references.

11. The system as claimed in claim 7, wherein the data labelling protocol comprises a set of inferences derived from screening and labelling a plurality of medical literature with suspected references to adverse drug reactions by the subject matter experts.

12. The system as claimed in claim 7, wherein the computing device is further configured to remove encoding errors and metatags from the search results; convert text in the search results into features capable of being inputted to the one or more machine learning models; and extract information from the search results for framing the plurality of data rules.
